(19) United States
(12) Patent Application Publication
(10) Pub. No.: US 2021/0196122 A1
(43) Pub. Date: Jul. 1, 2021

(54) PATIENT MONITOR SYSTEM AND PROCESS FOR ESTABLISHING A BLUETOOTH PAIRING OF A PATIENT MONITOR SYSTEM

(71) Applicant: Drägerwerk AG & Co. KGaA, Lübeck (DE)

(72) Inventors: Henning GERDER, Lübeck (DE); Roelof BERG, Lübeck (DE); Marke SPIESS, Hildesheim (DE); Bob KANG, Pu Dong New Area Shanghai (CN); Eveline ZHAO, Pu Dong New Area Shanghai (CN); Mike SONG, Shanghai (CN)

(21) Appl. No.: 17/057,890

(22) PCT Filed: Mar. 29, 2019
(86) PCT No.: PCT/EP2019/057972

§ 371 Date: Nov. 23, 2020

(30) Foreign Application Priority Data
May 24, 2018 (DE) 10 2018 004 201.5
Mar. 6, 2019 (DE) 10 2019 001 483.9

(51) Int. Cl. A61B 5/00; A61B 5/0205; H04W 4/80; H04W 76/10; H02J 7/00

(52) U.S. Cl. CPC A61B 5/002; A61B 5/02055; H04W 4/80; H04W 76/10; A61B 5/024; H02J 7/00032; A61B 2560/0456; A61B 2560/0214; H02J 7/0045

(57) ABSTRACT

A process, establishing Bluetooth pairing between a patient sensor (1) and a patient monitor (2), includes docking energy contacts (3) of the patient sensor with charging contacts (4) of a charging device (5) for charging a battery (6) of the patient sensor with an automatic detection of docking of the patient sensor with the charging device by a monitor detection device (7) of the patient monitor. The patient monitor is brought into a state of readiness for establishing Bluetooth pairing with the patient sensor. Patient sensor and charging device docking is automatically detected by a sensor detection device (8) of the patient sensor. The patient sensor is brought into a state of readiness for establishing Bluetooth pairing with the patient monitor. Bluetooth pairing between the patient sensor and the patient monitor is automatically established. A patient monitor system (20) is configured for carrying out the process.

### CROSS REFERENCE TO RELATED APPLICATIONS

This application is a United States National Phase Application of International Application PCT/EP2019/057972, filed Mar. 29, 2019, and claims the benefit of priority under 35 U.S.C. § 119 of German Applications 10 2018 004 201.5, filed May 24, 2018 and 10 2019 001 483.9, filed Mar. 6, 2019, the entire contents of which are incorporated herein by reference.

### TECHNICAL FIELD

The present invention pertains to a process for establishing a Bluetooth pairing between a patient sensor for detecting patient data and a patient monitor for outputting patient information on the basis of the detected patient data. The present invention further pertains to a patient monitor system with a patient sensor, with a patient monitor and with a charging device for charging a battery of the patient sensor.

### TECHNICAL BACKGROUND

Many different patient monitor systems for monitoring patients, e.g., in an intensive care unit or in a neonatal care unit of a hospital, are known. Patient monitor systems have one or more patient sensors for detecting patient parameters, e.g., the heart rate, respiration rate, body temperature or the like, as well as a patient monitor for outputting information on the patient, e.g., an optical display of the patient parameters determined on a display unit, acoustic or optical alarms or the like, on the basis of the detected patient parameters. An important function of a patient monitor system is the transmission of the determined patient parameters from the patient sensor to the patient monitor, in addition to the determination of the patient parameters and the outputting of the patient information. The transmission may take place, for example, completely, after which each determined patient parameter is passed on from the patient sensor to the patient monitor. As an alternative, a selective transmission of the patient parameters take place, e.g., patient parameters beyond specified threshold values, deviations from previously transmitted patient parameters or the like.

Various transmission systems are known for the transmission of the determined patient parameters. Especially reliable are wired transmission systems, because these guarantee a nearly trouble-free data transmission. In addition, wired transmission systems have an especially high level of data security, because the data transmission usually takes place via a data cable, which connects the patient sensor directly to the patient monitor. An unauthorized tapping of the data by third parties is possible with very complicated means only. Wired transmission systems do, however, have many drawbacks, e.g., that data cables and the corresponding connection plugs may be subject to a high level of wear and become easily damaged in case of daily use. Further, data cables have a comparatively high space requirement and may hide other objects, e.g., operating elements or display elements. In addition, it is possible in the case of data cables that connection plugs are unintentionally pulled out of the connection jackets and the data transmission is thus interrupted. Likewise, the position of the patient sensor may be changed due to the data cable by a movement of the patient, so that the patient sensor can no longer determine optimal patient parameters. There is a risk of strangulation due to data cables especially in case of very ill or especially young patients. Further, data cables represent an additional risk of infection, because these come regularly into contact with different patients and must therefore be disinfected. Finally, the freedom of movement and hence the comfort of the patient are restricted by data cables. The use of data cables is not therefore expedient in many applications.

An alternative data transmission device for transmitting the determined patient parameters from the patient sensor to the patient monitor is a Bluetooth connection. Bluetooth connections as radio-based data connections are sufficiently known from the state of the art. Bluetooth is a standardized protocol for data transmission. In Bluetooth, there is a master device, which is paired with no slave device, with one slave device or with a plurality of slave devices. The pairs or the pairing between two Bluetooth-capable devices is also called coupling or establishment of a Bluetooth pairing (Bluetooth pairing). The pairing is a first connection set-up between the Bluetooth-capable devices for authorizing a Bluetooth connection for the data exchange. Ratings of the devices are exchanged in order for devices paired with one another to be able to easily recognize each other and to be able to directly establish a Bluetooth connection with one another for the data exchange. A repeated pairing is not necessary any longer in most cases. To increase the security, an authorization is frequently performed during the pairing. For example, the input of a pin may be necessary in this connection. Various Bluetooth-based patient monitor systems are described in WO 2015/085332 A1, US 2016/0367190 A1 and CN 205334699 U.

Such processes for establishing a Bluetooth pairing have the drawback that they always must be initiated manually by a user. This is time-consuming and often requires a technical expertise, which is not necessary for the intended use of the devices paired with one another. Moreover, errors may occur in case of manually initiated Bluetooth pairings, so that a pairing is not performed correctly or it is carried out unknowingly between incorrect devices. Such an error remains undiscovered in the worst case, so that, for example, patient parameters of a first patient are transmitted to a patient monitor of a second patient and vice versa. Such a case is to be avoided by all means for the safety of the patients.

### SUMMARY

Based on this state of the art, a basic object of the present invention is to provide a process for establishing a Bluetooth pairing between a patient sensor and a patient monitor as well as to provide a patient monitor system with a patient sensor and with a patient monitor, which are free or at least partially free from these drawbacks of the state of the art. The object of the present invention is therefore to provide a process as well as a patient monitor system, which guarantee the establishment of a Bluetooth pairing between the patient sensor and the patient monitor in a simple as well as cost-effective manner. The above object is accomplished by a process for establishing a Bluetooth pairing between a patient sensor and a patient monitor with the features of the independent claim 1 as well as by a patient monitor system with a patient sensor and with a patient monitor with the features of the coordinate claim 6. Further features and details of the present invention appear from the subclaims, from the description and from the drawings. Features and details that are described in connection with the process according to the present invention are also valid in connection with the patient monitor system according to the present invention and vice versa, so that reference is and can always be made to the individual aspects of the invention concerning the disclosure of the individual aspects of the present invention.

According to a first aspect of the present invention, the object is accomplished by a process for establishing a Bluetooth pairing between a patient sensor for detecting as well as transmitting at least one patient parameter and a patient monitor for receiving the at least one detected patient parameter from the patient sensor as well as for outputting patient information concerning the at least one detected patient parameter.

The process has the following steps: docking of energy contacts of the patient sensor with charging contacts of a charging device for charging a battery of the patient sensor, automatic detection of the docking of the patient sensor with the charging device by means of a monitor detection device of the patient monitor, bringing the patient monitor into a state of readiness for establishing a Bluetooth pairing with the patient sensor, automatic detection of the docking of the patient sensor with the charging device by means of a sensor detection device of the patient sensor, bringing the patient sensor into a state of readiness for establishing a Bluetooth pairing with the patient monitor, and automatic establishing of a Bluetooth pairing between the patient sensor and the patient monitor.

A patient is defined within the framework of the patient invention as an acute ill patient, i.e., a human being who is already sick or a sick animal, as well as as a potential patient, i.e., a human being considered to be healthy or an animal considered to be healthy. A patient is accordingly especially a person to be monitored medically, e.g., an infant, a baby, a toddler, a child, a sick human being, an injured human being or an elderly person. The patient sensor is configured to detect at least one patient parameter and preferably a plurality of patient parameters, e.g., the presence of a heart beat, heart rate, body temperature, blood pressure or the like. As an alternative or in addition, the patient sensor may be configured to detect environmental parameters in the area around the patient, e.g., air temperature, oxygen level or the like. The patient sensor preferably has one or more acceleration sensors and/or one or more position sensors and/or one or more temperature sensors for this purpose. Moreover, the patient sensor has a Bluetooth module for transmitting patient information, for example, the at least one detected patient parameter, information derived herefrom or the like to the patient monitor. For charging the battery of the patient sensor, the latter has energy contacts, which can be brought into contact with charging contacts of the charging device for this purpose. The patient sensor is docked with the charging device in this state. The patient sensor preferably has a sensor housing, which protects components of the patient sensor, e.g., sensors, battery, printed circuit board, Bluetooth module or the like from external effects, for example, moisture, contamination or the like. The housing preferably has a watertight configuration. Further, the housing is preferably manufactured from an easy-to-clean and easy-to-disinfect material, especially a plastic, fiber composite or metal.

Provisions may be made according to the present invention for the patient sensor to have a fastening device, e.g., a magnetic fastener, a clamping fastener, a fastening strap or the like for temporary fixation to an article of clothing of the patient or directly on the patient.

The patient monitor is configured to receive the at least one detected patient parameter from the patient sensor. The patient monitor preferably likewise has to this end a Bluetooth module, which is configured to undergo a Bluetooth pairing with the Bluetooth module of the patient sensor for transmitting data, especially patient parameters. In addition, the patient monitor is configured to output at least one piece of patient information in connection with the at least one received patient parameter. The patient information may comprise, for example, qualitative and/or quantitative data on the heart rate, body temperature, blood pressure or the like. In addition or as an alternative, the patient information may take place in the form of a warning signal, e.g., in case of an excessively low heart rate or excessively high body temperature. A warning signal may be, for example, an optical warning signal, e.g., a warning light, a display on a display unit or the like, or an acoustic warning signal, e.g., a buzzer, a voice response or the like, or a combination thereof. The patient monitor preferably has a monitor housing, which protects components of the patient monitor, e.g., the Bluetooth module, printed circuit board or the like from external effects, e.g., moisture, contamination or the like. The housing preferably has a watertight configuration. The housing is preferably also made of an easy-to-clean and easy-to-disinfect material, especially a plastic, fiber composite or metal. The energy contacts of the patient sensor are brought into contact with charging contacts of the charging device during docking, so that an electrical current can flow between the energy contacts and the charging contacts for charging the battery of the patient sensor. Provisions may be made according to the present invention for energy contacts and charging contacts to have coils and for being configured for the inductive transmission of energy. “Bringing into contact” is defined in this case within the framework of the present invention as a relative arrangement of the coils, in which the energy transmission can take place between the coils. Such energy contacts have the advantage that these can be arranged within the sensor housing, so that the sensor housing may be configured as a fully closed housing.

The protection of the inner components is thus improved by the sensor housing. In addition, the fewer interruptions, partition lines, edges, shoulders or the like the sensor housing has, the easier it is to clean and to disinfect the sensor housing. The docking of the patient sensor with the charging device is detected automatically by means of the monitor detection device of the patient monitor. This may take place, for example, by the detection of a charging current for charging the battery. Accordingly, the monitor detection device may be configured, for example, as a part of the charging device. As an alternative or in addition, the monitor detection device may have a sensor, e.g., a magnetic sensor. The patient sensor preferably has to this end at least one magnet, which can be detected by the magnetic sensor, if the energy contacts are docked with the charging contacts. In addition or as an alternative, a push-button may be provided for detecting the docking of the patient sensor with the charging device. The push-button is preferably arranged at the charging device such that the push-button is actuated when the patient monitor is arranged at the charging device. The automatic detection of the docking can be carried out in this manner with simple means as well as in a cost-effective manner.

Prior to the automatic detection of the docking by the patient monitor, the latter is preferably in a secure state, in which the establishment of a Bluetooth pairing with the patient sensor is blocked. This has the advantage that the establishment of Bluetooth pairings does not take place arbitrarily. When the docking is detected automatically by the patient monitor, the latter is brought into the state of readiness for establishing the Bluetooth pairing with the patient sensor. A specific establishment of a Bluetooth pairing can thus be guaranteed. The docking of the patient sensor with the charging device is detected automatically by means of the sensor detection device of the patient sensor. This can take place, for example, by detecting a charging current for charging the battery. As an alternative or in addition, the sensor detection device may have a sensor, e.g., a magnetic sensor. The patient monitor preferably has to this end at least one magnet, which can be detected by the magnetic sensor when the energy contacts are docked with the charging contacts. The automatic detection of the docking can be carried out in this manner with simple means as well as in a cost-effective manner. The automatic detection of the docking of the patient sensor with the charging device by means of the monitor detection device preferably takes place simultaneously with the automatic detection of the docking of the patient sensor with the charging device by means of the sensor detection device. Before the automatic detection of the docking by the patient sensor, the latter is preferably in a secure state, in which the establishment of a Bluetooth pairing with the patient monitor is blocked. This has the advantage that the establishment of Bluetooth pairings does not take place arbitrarily. When the docking is detected by the patient sensor automatically, the patient sensor is brought into the state of readiness for establishing the Bluetooth pairing with the patient monitor. A specific establishment of a Bluetooth pairing can thus be guaranteed. The patient monitor is preferably brought into the state of readiness simultaneously with the bringing of the patient sensor into the state of readiness.

The initiation of the automatic establishment of the Bluetooth pairing may originate according to the present invention from the patient sensor, from the patient monitor or from both devices if these have been brought into the state of readiness. The automatic establishment of the Bluetooth pairing may take place, for example, without additional authorization corresponding to the Bluetooth standard according to a known Bluetooth pairing process. The Bluetooth pairing between the patient sensor and the patient monitor is preferably stored in the patient monitor, so that a Bluetooth connection can be established automatically when using the patient sensor in the Bluetooth reception range of the patient monitor without a repeated Bluetooth pairing being required.

The process according to the present invention for establishing a Bluetooth pairing between a patient sensor and a patient monitor has the advantage over conventional processes that a Bluetooth pairing can be established automatically with simple means as well as in a cost-effective manner, and triggering of the Bluetooth pairing only requires that the patient sensor and the charging device be brought physically together. It is avoided based on the dependence of the states of readiness on the automatic detection of the docking that incorrect devices would be paired with one another. A user of a patient monitor and a patient sensor can thus carry out the Bluetooth pairing without having to be aware of doing so. A simple docking of the patient sensor with the charging device is sufficient for this. A special technical training of the user in the field of the pairing process for pairing the patient sensor and the patient monitor is thus unnecessary.

It is preferred according to the present invention that the Bluetooth pairing between the patient sensor and the patient monitor is stored in the patient monitor, and the following process steps are carried out in the process: docking of energy contacts of at least one additional patient sensor with the charging contacts, automatic detection of the docking of the additional patient sensor with the charging device by means of the monitor detection device, bringing of the patient monitor into a state of readiness for establishing a Bluetooth pairing with the additional patient sensor, automatic detection of the docking of the additional patient sensor with the charging device by means of a sensor detection device of the additional patient sensor, bringing of the additional patient sensor into a state of readiness for establishing a Bluetooth pairing with the patient monitor, and automatic establishment of a Bluetooth pairing between the additional patient sensor and the patient monitor.

The Bluetooth pairing between the patient sensor and the at least one additional patient monitor is preferably likewise stored in the patient monitor. Provisions may be made in this connection according to the present invention for the previous Bluetooth pairing to be overwritten or deleted. This has the advantage that a user can always establish between which devices a Bluetooth pairing is present.

Further, the Bluetooth pairings between a plurality of patient sensors and the patient monitor are preferably preserved. This means that a Bluetooth pairing of a patient sensor with the patient monitor does not have to be automatically deleted when an additional patient sensor is paired with the patient monitor. Accordingly, the patient monitor has a Bluetooth pairing with at least two patient sensors. The process according to the present invention can preferably be carried out with at least one additional patient sensor. Up to four patient sensors are preferably paired in this manner with the patient monitor and the respective Bluetooth pairings are preferably stored in the patient monitor. This has the advantage that a Bluetooth connection can be automatically established when using the patient sensors in the Bluetooth reception range of the patient monitor, without a repeated Bluetooth pairing being required. In addition, a plurality of active Bluetooth connections can be established simultaneously between patient sensors and the patient monitor.

According to a preferred variant of the present invention, provisions may be made in a process for the patient sensor to transmit patient sensor information to the patient monitor via an information interface after the detection of the docking, and the patient monitor selects the patient sensor for the automatic establishment of the Bluetooth pairing on the basis of the transmitted patient sensor information. The information interface is preferably a data interface, which is configured for the wired data transmission. As an alternative, the information interface may be configured for wireless data transmission by means of a near field, especially according to the RFID technology. The patient sensor information may have, for example, a MAC address of the Bluetooth module of the patient sensor. It is preferred in this case that the initiation of the automatic establishment of the Bluetooth pairing is carried out by means of the patient monitor.

Based on the patient sensor information, the patient monitor is able to specifically select the patient sensor for the automatic establishment of the Bluetooth pairing, so that the selection of an incorrect patient sensor, which was, for example, brought at the same time into the state of readiness, is avoided. The transmission of patient sensor information has the advantage that the security in establishing the Bluetooth pairing can be improved with simple means as well as in a cost-effective manner.

The patient monitor preferably transmits after detection of docking via an information interface the patient monitor information to the patient sensor, and the patient sensor selects the patient monitor for the automatic establishment of the Bluetooth pairing on the basis of the transmitted patient monitor information. The information interface is preferably a data interface, which is configured for the wired data transmission. As an alternative, the information interface may be configured for the wireless data transmission by means of a near field, especially according to the RFID technology. The patient monitor information may have, for example, a MAC address of the Bluetooth module of the patient monitor. It is preferred in this case that the initiation of the automatic establishment of the Bluetooth pairing takes place by means of the patient sensor. The patient sensor is able on the basis of the patient monitor information to specifically select the patient monitor for the automatic establishment of the Bluetooth pairing, so that the selection of an incorrect patient monitor, which was brought, for example, simultaneously into the state of readiness, is avoided. The transmission of patient monitor information has the advantage that the security in establishing the Bluetooth pairing can be improved with simple means as well as in a cost-effective manner.

Further, a first charging contact of the charging device as well as a first energy contact of the patient sensor are preferably used as the information interface, the first energy contact contacting the first charging contact in an electrically conductive manner. The first charging contact and the first energy contact have a plurality of functions in this case. On the one hand, they are used to transmit electrical energy for charging the battery; in addition, they are used to transmit information. A data bit can thus be transmitted, for example, via the information interface. Such a solution can be embodied in an especially cost-effective manner. As an alternative, the charging current may be modulated such that more complex patient sensor information or patient monitor information or patient sensor information or patient monitor information comprising a plurality of bits is transmitted as well. This has the advantage that an individual code of the patient monitor or of the patient sensor is transmitted via the information interface, so that the automatic establishment of the Bluetooth pairing can be carried out specifically between the devices to be paired. The risk of a coupling by mistake with an incorrect device is thus ruled out or at least substantially reduced.

Provisions may be made according to the present invention for a device communication contact of the charging device and for a sensor communication contact of the patient sensor to be used as information interface, the device communication contact being different from the charging contacts. The sensor communication contact is preferably different from the energy contacts. The device communication contact and the sensor communication contact have only one function in this case, namely, the transmission of data. The charging of the battery may remain unaffected by the transmission of the data in this configuration. Further, the use of such an information interface has the advantage that device information, e.g., patient sensor information or patient monitor information, can be transmitted with simple means as well as in a cost-effective manner. The automatic establishment of the Bluetooth pairing can be carried out hereby in a specific manner between the devices to be paired, so that the risk of coupling with an incorrect device by mistake is thus ruled out or is at least substantially reduced.

According to a second aspect of the present invention, the object is accomplished by a patient monitor system. The patient monitor system has a patient sensor for detecting as well as for transmitting at least one patient parameter, a patient monitor for receiving the at least one detected patient parameter from the patient sensor via a Bluetooth connection as well as for outputting patient information concerning the at least one detected patient parameter and a charging device with charging contacts. The patient sensor has a battery for the power supply of the patient sensor as well as energy contacts for docking with the charging contacts of the charging device for charging the battery.

The patient monitor system is configured according to the present invention to carry out a process according to the first aspect of the present invention. A patient is defined within the framework of the present invention as a person who is to be monitored especially medically, e.g., a baby, a sick person, an injured person or an elderly person. The patient sensor is configured to detect at least one and preferably a plurality of patient parameters, e.g., the presence of a heart beat, heart rate, body temperature, blood pressure or the like. As an alternative or in addition, the patient sensor may be configured to detect environmental parameters in the area around the patient, e.g., air temperature, oxygen level or the like. The patient sensor preferably has for this purpose one or more acceleration sensors, one or more position sensors or one or more temperature sensors. Moreover, the patient sensor has a Bluetooth module for transmitting the at least one detected patient parameter to the patient monitor. To charge the battery of the patient sensor, the latter has energy contacts, which can be brought to this end into contact with charging contacts of the charging device. The patient sensor is docked in this state with the charging device. The patient sensor preferably has a sensor housing, which protects components of the patient sensor, e.g., sensors, battery, printed circuit board, Bluetooth module or the like, from external effects, e.g., moisture, contamination or the like. The housing preferably has a watertight configuration. Further, the housing is manufactured preferably from an easy-to-clean and easy-to-disinfect material, especially a plastic, a fiber composite or metal. Provisions may be made according to the present invention for the patient sensor to have a fastening device, e.g., a magnetic fastener, a clamping fastener, a fastening strap or the like, for temporary fixation to an article of closing of the patient or directly at the patient.

The patient monitor is configured to receive the at least one detected patient parameter from the patient sensor. The patient monitor preferably also has to this end a Bluetooth module, which is configured to undergo a Bluetooth pairing with the Bluetooth module of the patient sensor for the transmission of data, especially patient parameters. In addition, the patient monitor is configured to output at least one piece of patient information in connection with the at least one received patient parameter. The patient information may comprise, for example, qualitative or quantitative data on the heart rate, body temperature, blood pressure or the like.

In addition or as an alternative, the patient information may be given in the form of a warning signal, e.g., in case of an excessively low heart rate or excessively high body temperature. A warning signal may be, for example, an optical warning signal, e.g., a warning light, a display on a display device or the like, or an acoustic warning signal, e.g., a buzzer, a voice response or the like, or a combination thereof. The patient monitor preferably has a monitor housing, which protects components of the patient monitor, e.g., the Bluetooth module, printed circuit board or the like, from external effects, e.g., moisture, contamination or the like. The housing preferably has a water-tight configuration. Further, the housing is preferably manufactured from an easy-to-clean and easy-to-disinfect material, especially a plastic, fiber composite or metal. Energy contacts of the patient sensor can be brought into contact with charging contacts of the charging device such that an electrical current can flow between the energy contacts and the charging contacts for charging the battery of the patient sensor. Provisions may be made according to the present invention for energy contacts and charging contacts to have coils and to be configured for the inductive transmission of energy. The feature “can be brought into contact” is defined in this case within the framework of the present invention as the possibility of such a relative arrangement of the coils that the energy transmission can take place between the coils. Such energy contacts have the advantage that these can be arranged within the sensor housing, so that the sensor housing may be configured as a fully closed housing. The protection of the inner components by the sensor housing is thus improved. In addition, the fewer interruptions, partition lines, edges, shoulders or the like the sensor housing has, the easier it is to clean and to disinfect the sensor housing. All the advantages that were already described in connection with a process according to the first aspect of the present invention are obtained in the patient monitor system described. Accordingly, the patient monitor system according to the present invention has the advantage over conventional patient monitoring systems that a Bluetooth pairing can be established automatically with simple means as well as in a cost-effective manner, and it is only necessary to bring the patient sensor and the charging device physically together to trigger the Bluetooth pairing. Based on the dependence of the states of readiness on the automatic detection of docking, pairing of incorrect devices with one another is avoided. A user of the patient monitor and patient sensor can thus carry out the Bluetooth pairing without having to be aware of it. A simple docking of the patient sensor with the charging device is sufficient for this. A special technical training of the user in the process of pairing a patient sensor and a patient monitor is thus unnecessary.

It is preferred that the patient monitor system has at least one additional patient sensor, preferably a total of three or four patient sensors. A plurality of patient sensors have the advantage that the vital parameters of a plurality of patients can be displayed with one patient monitor.

The patient monitor system is preferably configured such that the Bluetooth pairing between the patient sensors and the patient monitor must be carried out only once and a telemetric Bluetooth connection (Bluetooth wireless connection) can be established automatically when at least one of the patient sensors is being operated in the Bluetooth reception range of the patient monitor. This has the advantage that the operation of the patient monitor system is made substantially easier.

The charging device is preferably integrated in the patient monitor. Further, components of the charging device are preferably arranged within a monitor housing of the patient monitor, and the charging contacts are preferably freely accessible from the outside, so that they can be brought into contact with the energy contacts. Further, a mount for mounting the patient sensor is preferably formed in the monitor housing. The mount is preferably configured such that the patient sensor can be arranged at the monitor housing in a positive-locking manner such that the energy contacts are in contact with the charging contacts. An integrated charging device has the advantage that the patient monitor system has a very compact configuration and data cables freely accessible from the outside are no longer necessary for coupling the charging device with the patient monitor.

In addition, it is preferred that the patient monitor has a monitor detection device for the automatic detection of the docking of the patient sensor with the charging device. The monitor detection device is configured, for example, for detecting a charging current for charging the battery. Accordingly, the monitor detection device may be configured, for example, as a part of the charging device. As an alternative or in addition, the monitor detection device may have a sensor, e.g., a magnetic sensor. The patient sensor preferably has to this end at least one magnet, which can be detected by the magnetic sensor when the energy contacts are docked with the charging contacts. A monitor detection device has the advantage that the docking of the patient sensor can be automatically detected with simple means as well as in a cost-effective manner.

Further, the patient sensor preferably has a sensor detection device for the automatic detection of the docking of the patient sensor with the charging device. The sensor detection device is configured, for example, for detecting a charging current for charging the battery. As an alternative or in addition, the sensor detection device may have a sensor, e.g., a magnetic sensor. The patient monitor preferably has for this purpose at least one magnet, which can be detected by the magnetic sensor when the energy contacts are docked with the charging contacts. A sensor detection device has the advantage that docking of the patient sensor can be automatically detected with simple means as well as in a cost-effective manner.

It is preferred according to the present invention that the patient monitor system has an information interface for the transmission of data between the patient sensor and the patient monitor, the information interface comprising a first charging contact of the charging contacts of the charging device and a first energy contact of the energy contacts of the patient sensor, the first charging contact and the first energy contact being configured for data transmission separately from the Bluetooth connection when the first charging contact and the first energy contact are in a touching contact with one another. The first charging contact and the first energy contact thus have a dual function, namely, the passage of the charging current for charging the battery of the patient sensor and the passing on of data. For example, a data bit can thus be transmitted via the information interface. Such a solution can be implemented in an especially cost-effective manner. As an alternative, the patient monitor system, especially the patient monitor and/or the patient sensor, are configured to modulate the charging current such that more complex patient monitor information and patient sensor information as well as patient monitor information and patient sensor information comprising a plurality of bits can also be transmitted via the information interface.

This has the advantage that an individual code of the patient monitor or patient sensor can be transmitted via the information interface, so that the automatic establishment of the Bluetooth pairing can be carried out in a specific manner between the devices to be paired. The risk of coupling by mistake with an incorrect device is thus ruled out or at least substantially reduced.

As an alternative, the patient monitor system has an information interface for the transmission of data between the patient sensor and the patient monitor, wherein the sensor communication contact of the information interface is arranged at the patient sensor and a device communication contact of the information interface is arranged at the charging device, wherein the sensor communication contact and the device communication contact are configured for the data transmission that is separate from the Bluetooth connection. The device communication contact is preferably different from the charging contacts and the sensor communication contact is different from the energy contacts. Further, the device communication contact and the sensor communication contact preferably have only one function, namely, the transmission of data. The charging of the battery can remain unaffected by the transmission of the data in this configuration. The patient sensor is preferably configured to transmit patient sensor information to the patient monitor via the information interface after the detection of the docking. The patient monitor is preferably configured to select the patient sensor for the automatic establishment of the Bluetooth pairing on the basis of the transmitted patient sensor information. The patient sensor information may have, for example, a MAC address of the Bluetooth module of the patient sensor. On the basis of the patient sensor information, the patient monitor is able to select the patient sensor specifically for the automatic establishment of the Bluetooth pairing, so that the selection of an incorrect patient sensor, which was, for example, brought simultaneously into the state of readiness, is avoided. The transmission of sensor information consequently has the advantage that the security of establishing the Bluetooth pairing is improved with simple means as well as in a cost-effective manner.

The sensor detection device is preferably configured to receive patient monitor information of the patient monitor. The patient monitor information may have, for example, a MAC address of the Bluetooth module of the patient monitor. Based on the patient monitor information, the patient sensor is able to specifically select the patient monitor for the automatic establishment of the Bluetooth pairing, so that the selection of an incorrect patient monitor, which was brought, for example, into the state of readiness simultaneously, can be avoided. The transmission of patient monitor information has the advantage that the security of establishing the Bluetooth pairing is improved with simple means as well as in a cost-effective manner.

It is further preferred that the monitor detection device is configured to receive patient sensor information of the patient sensor. The patient sensor information is preferably information by means of which the patient sensor can be unambiguously identified. The patient sensor information may have, for example, a MAC address of the Bluetooth module. Based on the patient sensor information, the patient monitor is able, for example, to select the patient sensor for the automatic establishment of the Bluetooth pairing in a specific manner, so that the selection of an incorrect patient sensor, which was brought, for example, into the state of readiness at the same time, can be avoided. The transmission of patient sensor information has the advantage that security is improved when establishing the Bluetooth pairing with simple means as well as in a cost-effective manner.

Provisions may be made according to a preferred variant of the present invention for the information interface to be configured such that a data transmission can only take place between the sensor communication contact and the device communication contact when the sensor communication contact and the device communication contact are in a touching contact with one another. This has the advantage that it is necessary to bring the patient sensor and the charging device physically together in order to establish the communication. The risk of mixing up devices and hence the risk of the unintended pairing of incorrect devices can thus be prevented with simple means as ell as in a cost-effective manner.

Further measures improving the present invention appear from the following description of some exemplary embodiments of the present invention, which are shown in the figures. All the features and/or advantages, including design details and arrangements in space, which appear from the claims, from the description or from the drawings, may be essential for the present invention both in themselves and in the different combinations. The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which preferred embodiments of the invention are illustrated.

### BRIEF DESCRIPTION OF THE DRAWINGS

In the drawings:

FIG. 1 is a schematic side view of a preferred first embodiment of a patient monitor system according to the present invention;

FIG. 2 is a schematic side view of a preferred second embodiment of a patient monitor system according to the present invention;

FIG. 3 is a schematic side view of a preferred third embodiment of a patient monitor system according to the present invention;

FIG. 4 is a flow chart of a preferred embodiment of the process according to the present invention; and

FIG. 5 is a process diagram of another preferred embodiment of the process according to the present invention.

### DESCRIPTION OF PREFERRED EMBODIMENTS

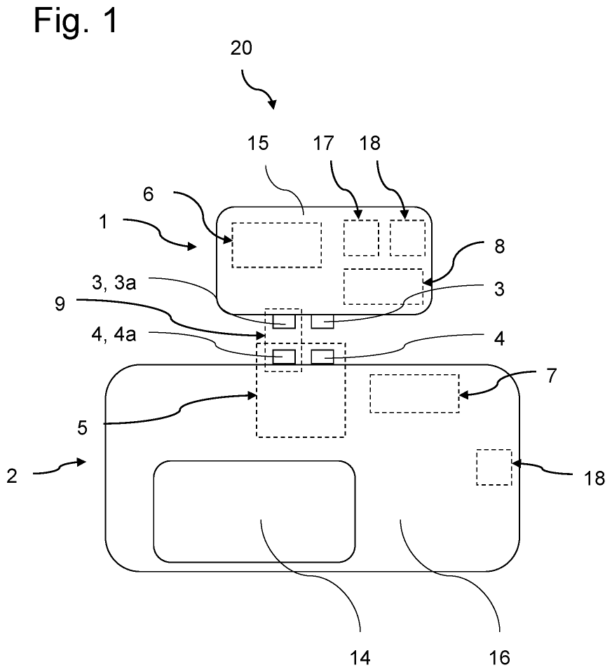

Referring to the drawings, elements having the same function and mode of operation are always designated by the same reference numbers in FIGS. 1 through 5.

FIG. 1 schematically shows a preferred first embodiment of a patient monitor system 20 according to the present invention in a side view. The patient monitor system 20 has a patient sensor 1 with a sensor housing 15 and a patient monitor 2 with a monitor housing 16.

The patient sensor 1 has a battery 6 for the power supply, a sensor 17 for detecting a patient parameter, especially a heart rate or body temperature, and a Bluetooth module 18. In addition, the patient sensor 1 has two energy contacts 3 for charging the battery 6. More than one patient sensor 1 may be provided as well. The patient monitor 2 has a charging device 5 with two charging contacts 4 for charging the battery 6. It is necessary for this to bring the energy contacts 3 into contact with the charging contacts 4. This operation is also called docking within the framework of the present invention. An undocked state is shown in FIG. 1. Further, the patient monitor 2 has a display 14 for displaying patient data, e.g., heart rate, body temperature or the like, as well as a Bluetooth module 18.

The patient sensor 1 has, in addition, a sensor detection device 8 for detecting the docking of the energy contacts 3 with the charging contacts 4. The patient monitor 2 correspondingly has a monitor detection device 7 for detecting the docking of the energy contacts 3 with the charging contacts 4. Further, the patient monitor system 20 has an information interface 9, which is composed of a first energy contact 3a of the energy contacts 3 and a first charging contact 4a of the charging contacts 4. The monitor detection device 7 and the sensor detection device 8 are preferably coupled with the information interface 9. The detection of docking can be carried out in this case via the information interface 9, especially when such a data transmission can only take place in the docked state.

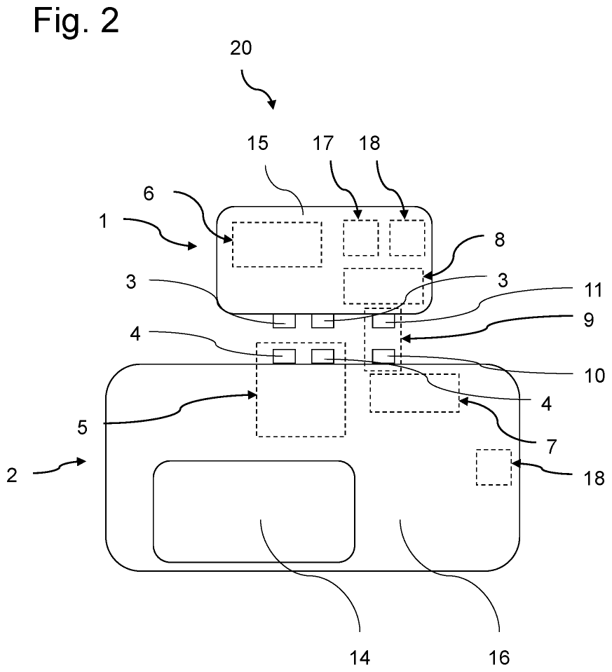

FIG. 2 schematically shows a preferred second embodiment of a patient monitor system 20 according to the present invention in a side view. The second embodiment differs from the first embodiment in the feature that the information interface 9 comprises in the second exemplary embodiment a sensor communication contact 11 of the patient sensor 1 and a device communication contact 10 of the patient monitor 2, the device communication contact 10 being different from the charging contact 4 and the sensor communication contact 11 being different from the energy contacts 3.

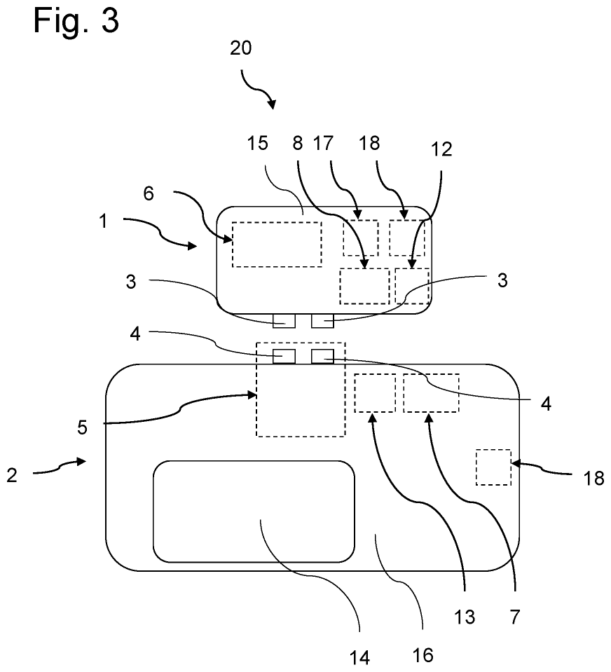

FIG. 3 schematically shows a preferred third embodiment of a patient monitor system 20 according to the present invention in a side view. The third embodiment differs from the first embodiment in the feature that no information interface 9 is present. In addition, the first patient sensor 1 has a sensor magnet 12 and the patient monitor 2 has a monitor magnet 13.

The sensor detection device 8 is configured in this exemplary embodiment to detect the monitor magnet 13 and the monitor detection device 7 is configured to detect the sensor magnet 12. The automatic detection of the docking of the patient sensor 1 with the charging device 5 can easily be carried out in this manner by the patient sensor 1 and by the patient monitor 2.

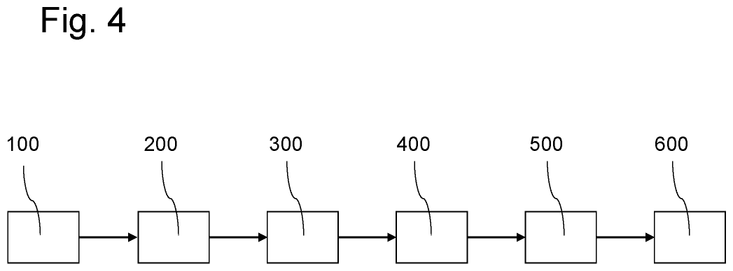

FIG. 4 schematically shows a flow chart of a preferred embodiment of the process according to the present invention. The energy contacts 3 of the patient sensor 1 are docked in a first process step 100 with charging contacts 4 of the charging device 5 for charging the battery 6 of the patient sensor 1. This operation may also be called docking of the patient sensor 1 with the charging device 5. In a second process step 200, the monitor detection device 7 of the patient monitor 2 automatically detects the docking of the patient sensor 1 with the charging device 5. This may be carried out, for example, by detecting a sensor magnet 12 of the patient sensor 1, a charging current through the charging device 5 or data transmitted via information interface 9. In a third process step 300, the patient monitor 2 is brought into a state of readiness for establishing a Bluetooth pairing with the patient sensor 1. In a fourth process step 400, the sensor detection device 8 of the patient sensor 1 detects the docking of the patient sensor 1 with the charging device 5 automatically. This may be carried out, for example, by detecting a monitor magnet 13 of the patient monitor 2, a charging current to the battery 6 or of data transmitted via the information interface. In a fifth process step 500, the patient sensor 1 is brought into a state of readiness for establishing a Bluetooth pairing with the patient monitor 2. The second process step 200 and the fourth process step 400 preferably take place simultaneously or at least essentially at the same time. Further, the third process step 300 and the fifth process step 500 preferably take place simultaneously or at least essentially simultaneously. In a sixth process step 600, a Bluetooth pairing is established automatically between the patient sensor 1 and the patient monitor 2 by means of the Bluetooth modules 18 of the patient sensor 1 and of the patient monitor 2. The patient sensor 1 and the patient monitor 2 are now paired with one another, so that a secure data transmission, especially a transmission of patient parameters from the patient sensor 1 to the patient monitor 2 can take place.

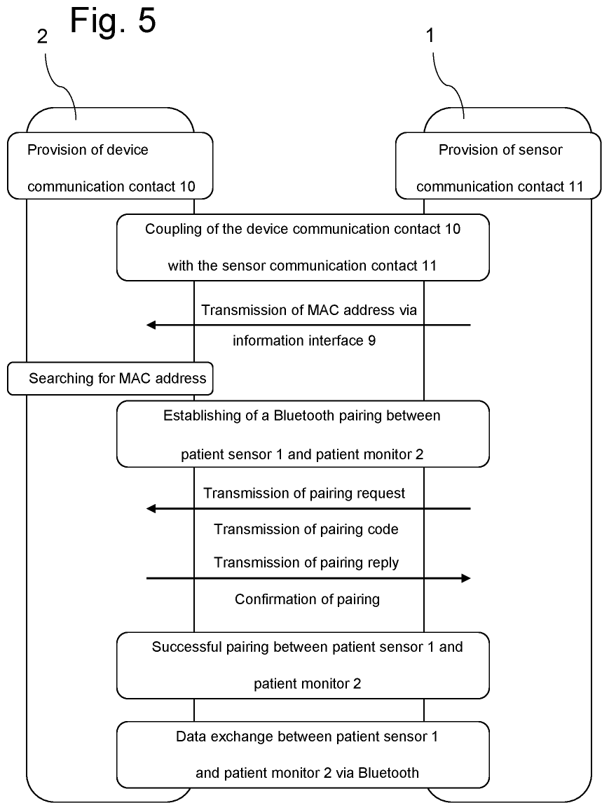

FIG. 5 schematically shows another preferred embodiment of the process according to the present invention in a process diagram. A possible communication between the patient sensor 1 and the patient monitor 2 is shown in the process diagram. The patient monitor 2 provides at first the device communication contact 10 of the information interface 9 and the patient sensor 1 provides the sensor communication contact 11 of the information interface 9. The information interface 9 is preferably configured for both GPIO and UART TX communication. The device communication contact 10 may transmit to this end, for example, an on/off signal, which can be detected by the sensor communication contact 11 after successful mechanical coupling of the patient sensor 1 with the patient monitor 2 and of the sensor communication contact 11 with the device communication contact 10. The patient sensor 1 then transmits its Bluetooth MAC address via the information interface 9 to the patient monitor 2. The patient monitor 2 searches for the MAC address in its environment and goes into a state of readiness for pairing with the patient sensor 1. The patient sensor 1 then transmits a BT pairing request to the patient monitor 2. The BT pairing request is preferably sent via the Bluetooth module of the patient sensor 1.

Provisions may be made for increased security for the patient sensor 1 additionally to transmit a pairing code via the information interface 9 to the patient monitor 2. The patient monitor 2 recognizes the MAC address of the patient sensor 1 and transmits a pairing reply with a pairing confirmation to the patient sensor 1. The transmission of the pairing reply preferably takes place via the Bluetooth module of the patient monitor 2. In case the patient sensor 1 has additionally transmitted the pairing code via the information interface 9 to the patient monitor 2, the patient monitor 2 can transmit the pairing code via the Bluetooth module of the patient monitor 2 to the patient sensor 1. A successful pairing has thus taken place between the patient sensor 1 and the patient module 2. A data transmission or a data exchange via Bluetooth may subsequently take place.

While specific embodiments of the invention have been shown and described in detail to illustrate the application of the principles of the invention, it will be understood that the invention may be embodied otherwise without departing from such principles.

1. A process for establishing a Bluetooth pairing between a patient sensor for detecting as well as sending at least one patient parameter and a patient monitor for receiving the at least one detected patient parameter from the patient sensor as well as for outputting patient information concerning the at least one detected patient parameter, the process comprising the steps of:
docking energy contacts of the patient sensor with charging contacts of a charging device for charging a battery of the patient sensor;
automatically detecting the docking of the patient sensor with the charging device by means of a monitor detection device of the patient monitor;
bringing the patient monitor into a state of readiness for establishing a Bluetooth pairing with the patient sensor;
automatically detecting the docking of the patient sensor with the charging device by means of a sensor detection device of the patient sensor;
bringing the patient sensor into a state of readiness for establishing a Bluetooth pairing with the patient monitor; and
automatically establishing a Bluetooth pairing between the patient sensor and the patient monitor.

2. A process in accordance with claim 1, wherein the Bluetooth pairing between the patient sensor and the patient monitor is stored in the patient monitor, and wherein the process further comprises the steps of:
docking energy contacts of an additional patient sensor with the charging contacts;
automatically detecting the docking of the additional patient sensor with the charging device by means of the monitor detection device;
bringing the patient monitor into a state of readiness for establishing a Bluetooth pairing with the additional patient sensor;
automatically detecting the docking of the additional patient sensor with the charging device by means of a sensor detection device of the additional patient sensor;
bringing the additional patient sensor into a state of readiness for establishing a Bluetooth pairing with the patient monitor; and
automatically establishing a Bluetooth pairing between the additional patient sensor and the patient monitor.

3. A process in accordance with claim 2, wherein the Bluetooth pairings between a plurality of patient sensors and the patient monitor are preserved.

4. A process in accordance with claim 1, wherein:
the patient sensor transmits patient sensor information to the patient monitor after the detection of the docking via an information interface; and
the patient monitor selects the patient sensor for automatically establishing the Bluetooth pairing on the basis of the transmitted patient sensor information.

5. A process in accordance with claim 1, wherein:
the patient monitor transmits patient monitor information to the patient sensor after the detection of the docking via an information interface; and
the patient sensor selects the patient monitor for automatically establishing the Bluetooth connection on the basis of the transmitted patient monitor information.

6. A process in accordance with claim 4, wherein:
a first charging contact of the charging device as well as a first energy contact of the patient sensor are used as the information interface; and
the first energy contact contacts the first charging contact in an electrically conductive manner.

7. A process in accordance with claim 4, wherein:
a device communication contact of the charging device and a sensor communication contact of the patient sensor are used as the information interface; and
the device communication contact is different from the charging contacts.

8. A patient monitor system comprising:
a patient sensor configured to detect and transmit at least one patient parameter;
a patient monitor configured to receive the at least one detected patient parameter from the patient sensor via a Bluetooth connection as well as for outputting patient information concerning the at least one detected patient parameter; and
a charging device with charging contacts, wherein the patient sensor has a battery for the power supply of the patient sensor as well as energy contacts for docking with the charging contacts of the charging device for charging the battery, the patient monitor comprises a monitor detection device configured to automatically detect a docking of the patient sensor with the charging device, the patient sensor comprises a sensor detection device configured to automatically detect the docking of the patient sensor with the charging device, and the patient monitor system is configured to
dock energy contacts of the patient sensor with the charging contacts of the charging device to charge the battery of the patient sensor;
automatically detect the docking of the patient sensor with the charging device by means of the monitor detection device of the patient monitor;
bring the patient monitor into a state of readiness for establishing a Bluetooth pairing with the patient sensor;
automatically detect the docking of the patient sensor with the charging device by means of the sensor detection device of the patient sensor;
bring the patient sensor into a state of readiness for establishing a Bluetooth pairing with the patient monitor; and
automatically establish a Bluetooth pairing between the patient sensor and the patient monitor.

9. A patient monitor system in accordance with claim 8, wherein the patient monitor system has at least one additional patient sensor.

10. A patient monitor system in accordance with claim 8, wherein the charging device is integrated in the patient monitor.

11-12. (canceled)

13. A patient monitor system in accordance with claim 8, further comprising: an information interface for the transmission of data between the patient sensor and the patient monitor, wherein the information interface comprises a first charging contact of the charging contacts of the charging device and a first energy contact of the energy contacts of the patient sensor, wherein the first charging contact and the first energy contact are configured for data transmission that is separate from the Bluetooth connection when the first charging contact and the first energy contact are in touching contact with one another.

14. A patient monitor system in accordance with claim 8, further comprising an information interface for transmitting data between the patient sensor and the patient monitor, wherein a sensor communication contact of the information interface is arranged at the patient sensor and a device communication contact of the information interface is arranged at the charging device, wherein the sensor communication contact and the device communication contact are configured for data transmission that is separate from the Bluetooth connection.

15. A patient monitor system in accordance with claim 14, wherein the information interface is configured such that a data transmission between the sensor communication contact and the device communication contact can only take place if the sensor communication contact and the device communication contact are in touching contact with one another.

16. A patient monitor system in accordance with claim 8, wherein the Bluetooth pairings between a plurality of patient sensors and the patient monitor are preserved.

17. A process in accordance with claim 5, wherein:
a first charging contact of the charging device as well as a first energy contact of the patient sensor are used as the information interface; and
the first energy contact contacts the first charging contact in an electrically conductive manner.

18. A process in accordance with claim 4, wherein:
a device communication contact of the charging device and a sensor communication contact of the patient sensor are used as the information interface; and
the device communication contact is different from the charging contacts.

19. A patient monitor system in accordance with claim 8, wherein:
the patient sensor transmits patient sensor information to the patient monitor after the detection of the docking via an information interface; and
the patient monitor selects the patient sensor for automatically establishing the Bluetooth pairing on the basis of the transmitted patient sensor information.

20. A patient monitor system in accordance with claim 8, wherein:
the patient monitor transmits patient monitor information to the patient sensor after the detection of the docking via an information interface; and
the patient sensor selects the patient monitor for automatically establishing the Bluetooth connection on the basis of the transmitted patient monitor information.

21. A patient monitor system in accordance with claim 19, wherein:
a first charging contact of the charging device as well as a first energy contact of the patient sensor are used as the information interface; and
the first energy contact contacts the first charging contact in an electrically conductive manner.

22. A patient monitor system in accordance with claim 19, wherein:
a device communication contact of the charging device and a sensor communication contact of the patient sensor are used as the information interface; and
the device communication contact is different from the charging contacts.

23. A patient monitor system in accordance with claim 20, wherein:
a first charging contact of the charging device as well as a first energy contact of the patient sensor are used as the information interface; and
the first energy contact contacts the first charging contact in an electrically conductive manner.

24. A patient monitor system in accordance with claim 20, wherein:
a device communication contact of the charging device and a sensor communication contact of the patient sensor are used as the information interface; and
the device communication contact is different from the charging contacts.
